(19) United States
(12) Patent Application Publication
(10) Pub. No.: US 2026/0215459 A1
(43) Pub. Date: Jul. 30, 2026

(54) Process of Encapsulating Oils, Product Obtained and Related Uses

(71) Applicant: PROMOTORA DE INNOVACION EN BIOTECNOLOGIA S.A.S., Bucaramanga, Santander (CO)

(72) Inventors: Blanca Cecilia MARTÍNEZ MORALES, Bucaramanga (CO); Loufrantz Jaffer PARRA MENDEZ, Bucaramanga (CO); Alvaro José URIBE SERRANO, Bucaramanga (CO)

(73) Assignee: PROMOTORA DE INNOVACION EN BIOTECNOLOGIA S.A.S., Bucaramanga, Santander (CO)

(21) Appl. No.: 19/039,963

(22) Filed: Jan. 29, 2025

(51) Int. Cl. A23K 40/30; A23K 10/14; A23K 20/105; A23K 20/111; A23K 20/163; A23K 40/35; A23K 50/10; A23K 50/30; A23K 50/75; C11B 9/00; C12N 9/26

(52) U.S. Cl. CPC A23K 40/30; A23K 10/14; A23K 20/105; A23K 20/111; A23K 20/163; A23K 40/35; A23K 50/10; A23K 50/30; A23K 50/75; C11B 9/0003; C12N 9/2414; C12Y 302/01001

(57) ABSTRACT

A formulation consisting of a starch containing amylose concentrations between 60% and 80% and amylopectin concentrations between 20% and 40% and may include water, an alpha amylase allowing α-1-4 enzymatic hydrolysis, an essential oil composition, sodium benzoate, potassium sorbate, anhydrous citric acid taken from a natural source as a pH regulator, optionally a high-methoxylated rapid citrus pectin, a surfactant—or mixture of surfactants—to regulate HLB.

### FIELD OF THE INVENTION

The present patent application relates to a process for the microencapsulation or nanoencapsulation of essential oils—particularly processes incorporating spray-drying stages—, products obtained and uses thereof.

### BACKGROUND OF THE INVENTION

Aromatic plants have long been used for their preservative and medicinal properties and to give aroma and flavor to foods. Its properties are partly attributed to essential oils. The term “essential oil” was first used in the 16th century by Paracelsus, who called the effective component of a medicine the “quinta essentia.” GUENTHER, ERNEST. The Essential Oils, D. VAN NOSTRAND COMPANY, INC. 1959. By the mid-20th century, the role of essential oils had been reduced to their use in perfumes, cosmetics, and food flavorings.

Essential oils are a diverse group of natural aromatic compounds isolated primarily from non-woody plant materials. They contain terpenoids, especially monoterpenes (C10), sesquiterpenes (C15), and diterpenes (C20), along with a variety of aliphatic (low molecular weight) hydrocarbons, acids, alcohols, aldehydes, and 2-4 esters. The main components are found in much higher concentrations than other components, which are only present in trace amounts. The main components determine their main activity and, due to their versatility, essential oils have multiple properties, such as bactericidal, fungicidal, antioxidant, virucidal or anticancer properties. Several techniques can be used to extract essential oils from different parts of the aromatic plant, such as water or steam distillation, solvent extraction, pressurized extraction, supercritical fluid extraction, and subcritical water extraction. See, Edris, Amr. Pharmaceutical and Therapeutic Potentials of Essential Oils and Their Individual Volatile Constituents: A Review. PHYTOTHERAPY RESEARCH Phytother. Res. 21 308-323 (2007), incorporated by reference herein.

Encapsulation has been widely used for the protection, selective delivery and enhancement of the biological functions of bioactive compounds. For example, carvacrol was microencapsulated in calcium alginate to reduce its early absorption in the gastrointestinal tract after oral administration, thus retaining its potential antibacterial activity for the small intestine. See, Wang, Q., Gong, J., Huang, X., Yu, H. and Xue, F. (2009), In vitro evaluation of the activity of microencapsulated carvacrol against Escherichia coli with K 88 pili. Journal of Applied Microbiology, 107: 1781-1788, https://doi.org/10.1111/j.1365-2672.2009.04374, incorporated by reference herein. Encapsulation not only provides controlled release, but also increases the bioavailability of certain compounds. For example, peppermint essential oil or carvacrol and eugenol have been nano encapsulated for such purposes. See, Liang R, Xu S, Shoemaker CF, Li Y, Zhong F, Huang Q. Physical and antimicrobial properties of peppermint oil nanoemulsions. J Agric Food Chem. 2012 Aug 1;60(30):7548-55. doi:10.1021/jf301129k. Epub 2012 Jul 19. PMID: 22,746,096 and Terjung, Nino: Löffler, Myriam; and Gibis, Monika; Hinirisch, Jörg; Weiss, Jochen. Influence of droplet size on the efficacy of oil-in-water emulsions loaded with phenolic antimicrobials. Food & Function. 2012 3, 290-301; both incorporated by reference herein.

Essential oils have volatile components that are sensitive to oxygen, light, moisture, and heat. Their encapsulation is a growing field due to the need to preserve their bioactive properties and protect them from degradation, as well as to improve their effectiveness in different applications. Encapsulating essential oils provides significant advantages in terms of stability, bioavailability, and release control.

Essential oils are highly susceptible to oxidation when exposed to oxygen and light, which can reduce their effectiveness. By encapsulating them, a barrier is created to protect them, prolonging their shelf life and maintaining their biological activity for a longer period of time, as well as improving their solubility and bioavailability.

Essential oils are hydrophobic by nature, which makes them difficult to dissolve in aqueous media. Encapsulation, especially in systems such as nanoemulsions and liposomes, can improve dispersion in aqueous media, facilitating their application in products such as beverages and pharmaceutical solutions. A key aspect of encapsulation is the ability to control the release of essential oils. Depending on the encapsulating material used, oils can be released in a sustained manner, which is crucial in applications such as animal feed, where a gradual release optimizes the absorption and utilization of active nutrients.

Microencapsulation uses carriers such as maltodextrin, gum arabic, and proteins, which form a protective layer around the essential oils. These capsules protect the oils from thermal degradation and oxidation. They are widely used in the food and cosmetics industry. See, V. gr. Madene, A., Jacquot, M., Scher, J. and Desobry, S. (2006), Flavour encapsulation and controlled release—a review. International Journal of Food Science & Technology, 41: 1-21. https://doi.org/10.1111/j.1365-2621.2005.00980.x, incorporated by reference herein. In nanoemulsions, the oil droplets are between 20-200 nanometers in size. Essential oils encapsulated in nanoemulsions are used in applications where high bioavailability and water solubility are required. Due to their size, nanoemulsions have high kinetic stability and can prevent coalescence of oil droplets. Different studies have shown the advantages and uses of one technique or the other. See, McClements, David Julian (2012). Nanoemulsions versus microemulsions: Terminology, differences, and similarities. Soft Matter, 8(6), 1719-1729. DOI: https: //doi. org/10.1039/C2SM06903B, incorporated by reference herein.

Liposomes, for their part, are vesicles formed by lipid bilayers that can encapsulate both hydrophilic and hydrophobic compounds. Essential oils encapsulated in liposomes are released in a controlled manner, which is useful in therapeutic and cosmetic applications. In addition, liposomes can be designed to release the encapsulated contents gradually, which maximizes their effectiveness. See, Mozafari, M. & Johnson, Chad & Hatziantoniou, Sophia & Demetzos, Costas. (2008). Nanoliposomes and Their Applications in Food Nanotechnology. Journal of liposome research. 18. 309-27. 10.1080/08982100802465941, incorporated by reference herein.

Cyclodextrins—cyclic oligosaccharide compounds—can also encapsulate essential oils within their structure, which improves water solubility and thermal stability. These inclusions are used in food and pharmaceutical products where it is necessary to increase the solubility of essential oils without affecting their organoleptic properties. See, Martin Del Valle, E.M. Cyclodextrins and their uses: a review, Process Biochemistry, Volume 39 Issue 9, 2004 Pages 1033-1046, ISSN 1359-5113, https://doi.org/10.1016/S0032-9592(03)00258-9, incorporated by reference herein.

Spray drying is another common method of encapsulating essential oils. It consists of spraying an emulsion of the essential oil in a drying chamber, where the solvent evaporates and a solid capsule is formed to protect the oil. It is a widely used method in the food industry due to its low cost and high efficiency. Gharsallaoui, Adem; Roudaut, Gaëlle; Chambin, Odile; Voilley, Andrée; Saurel, Rémi. Applications of spray-drying in microencapsulation of food ingredients: An overview, Food Research International, Volume 40 Issue 9, 2007 Pages 1107-1121, ISSN 0963-9969, https://doi.org/10.1016/j.foodres.2007.07.004, incorporated by reference herein.

The application of essential oil encapsulation techniques in animal feed has gained importance in recent years due to the growing demand for natural and effective solutions that improve animal health and productivity. Some of the main benefits are:
Improved stability. Essential oils, when administered directly, are highly unstable. They may lose their bioactive properties due to oxidation or volatilization during food storage or processing. Encapsulation techniques, such as microencapsulation and spray drying, make it possible to isolate the essential oil from the environment, protecting it from oxidation and preserving its properties for longer periods. This is essential to ensure that the oil retains its antimicrobial and antioxidant activity, which is key to improving animal health.
Controlled release: A fundamental aspect of encapsulation is the ability to design systems that allow sustained release of essential oils in the digestive tract of animals. This optimizes the absorption of the active compounds, maximizing their benefits. The controlled release of essential oils encapsulated in polymeric matrices or maltodextrin nanoparticles, for example, allows gradual release of the oils in response to specific stimuli such as stomach pH, which is essential to improve the bioavailability of essential oils and ensure that they act effectively along the gastrointestinal tract.
Antimicrobial and antiparasitic effectiveness. Several studies show that encapsulated essential oils, such as oregano oil and thymol, have significant antimicrobial effects in animal feed. By encapsulating these oils in systems such as nanoemulsions or microparticles, their effects against pathogenic bacteria can be concentrated without adversely affecting the beneficial microbiota. These systems are useful in reducing the use of antibiotics in livestock farming, providing a more natural and sustainable alternative for the control of infectious diseases in animals.

By encapsulating essential oils in forms such as nanoparticles or complexes with cyclodextrins, animals have been shown to improve their feed conversion. This means that they use feed nutrients more efficiently, which, in turn, improves growth and production performance of animals such as poultry and pigs. These allow the active compounds of the essential oils to act for a prolonged time in the intestine, which not only improves nutrient absorption, but also boosts immune function and reduces gastrointestinal infections.

Some examples of its use are related to the encapsulation of essential oils in nanoemulsions. Nanoemulsions have been used to encapsulate essential oils such as thymol, carvacrol, and eugenol. These formulations allow greater penetration of the active compounds into the cells of the intestinal tract, improving digestion and absorption of nutrients. In addition, nanoemulsions exhibit excellent dispersion in aqueous media, making them ideal for administration in liquid or premixed foods. See, McClements, David Julian (2012). Nanoemulsions versus microemulsions: Terminology, differences, and similarities. Soft Matter, 8(6), 1719-1729. DOI: https://doi.org/10.1039/C2SM06903B, incorporated by reference herein.

Microencapsulations have also been worked on using maltodextrin and cassava starch derivatives. These encapsulants provide a protective matrix for essential oils, improving their stability and facilitating their incorporation into balanced feed for poultry and pigs. An additional benefit is that these polymers are easily digestible, which allows a controlled release of the essential oils during the digestive process. See, Madene, A., Jacquot, M., Scher, J. and Desobry, S. (2006), Flavour encapsulation and controlled release—a review. International Journal of Food Science & Technology, 41:1-21. https://doi.org/10.1111/j.1365-2621.2005.00980.x, incorporated by reference herein.

Cyclodextrins, especially β-cyclodextrins, are used to encapsulate essential oils, improving their solubility and stability. These inclusion complexes are particularly useful in the feeding of young animals, which require a more controlled release of nutrients. Cyclodextrins protect essential oils from degradation in the rumen of ruminants, allowing them to reach the small intestine intact, where they are released and absorbed. See, Martin Del Valle, E.M. Cyclodextrins and their uses: a review, Process Biochemistry, Volume 39 Issue 9, 2004 Pages 1033-1046, ISSN 1359-5113, https://doi.org/10.1016/S0032-9592(03)00258-9, incorporated by reference herein.

Different developments show how attempts have been made to encapsulate essential oils at the industrial level, including processes involving spraying. However, nothing in the available literature allows us to identify industrial processes associated with nano-or microencapsulation with precise steps for their industrial commercialization. Some documents, for example, include WO2024130455A1—which presents a microencapsulated repellent and/or flavoring formulation for the agroforestry and wood industry—, WO2022101613A1—which refers to a fungicidal composition from essential oils—, CN101519624A—refers to the microencapsulation of a cinnamon essential oil—or US2024197618A1—microencapsulation of a polysander oil—; all four incorporated by reference herein.

Therefore, the present invention is related to a standardized industrial process specially designed to work essential oils useful to be employed mainly in animal feed.

### SUMMARY OF THE INVENTION

The present invention relates a process for encapsulating essential oils from an initial formulation with starches with amylose concentrations between 60 and 80% and from 20 to 40% amylopectin and mixtures of essential oils which may include Eugenol, Betacaryophyllene, Thymol, p-cymene, Terpinene, Thymyl acetate, ß-myrcene, Trans-ß-Caryophyllene, Methyl thymyl ester, Carvacrol, among others. The products are formulated in the following quantities:
45-55% starch
35-45% water
0-0.1% alpha-amylase
5-15% mixture of essential oils
0.01-0.10% benzoate
0.01-0.10% sorbate
0-0.05% citric acid
0-0.5% citrus pectin
0-10% organic acids selected from fumaric, formic, propionic, lactic, butyric, or a mixture thereof

The process focuses on introducing water into a reactor under stirring at a range between 95 and 105 rpm, adding starch and alpha-amylase and raising the temperatures in steps, first between 45 and 55° C., then between 65 and 75° C., and, finally, between 85 and 95° C., keeping the temperature constant in each range for 10 to 25 minutes, developing a recirculation of the mixture. The temperature is then lowered to 65 and 75° C., more starch and alpha-amylase are added, and the previous steps of temperature escalation and recirculation are repeated. The temperature is once again lowered to 65 and 75° C. and the process of temperature escalation and recirculation is repeated for the last time. The formulated acids, benzoate, and sorbate are added. The mixture is then introduced into a new reactor under stirring, where the essential oils are also incorporated with controlled stirring and, then, the mixture is taken to spray drying to obtain encapsulated essential oils.

### DETAILED DESCRIPTION OF THE INVENTION

The present invention describes a process for encapsulating nano or microparticles of essential oils in a starch-based vehicle. The process begins with the preparation of a formulation consisting of
A starch having amylose concentrations between 60% and 80% and amylopectin concentrations between 20% and 40%. In particular, the following can be used:
   Cassava starch containing between 70%-80% amylose and 20%-30% amylopectin
   Corn and cassava starch containing between 65%-75% amylose and 25%-35% amylopectin
   Potato starch containing between 70%-80% amylose and 20-30% amylopectin
   Wheat starch containing 70%-75% amylose and 25%-30% amylopectin
   Rice starch containing between 60%-80% amylose and 20%-40% amylopectin
   Green banana starch between 70%-75% amylose and 25%-30% amylopectin
Water
An alpha amylase allowing α-1-4 enzymatic hydrolysis.
A composition of essential oils that may include the following components or mixtures thereof
   Eugenol
   Betacaryophyllene
   Thymol
   p-cymene
   γ-Terpinene
   Thymyl acetate
   ß-myrcene
   Trans-ß-Caryophyllene
   Methyl thymyl ester
   Carvacrol
   p-cymene
   Geranial
   Neral
   Geraniol
   1.8 cineol
   Limonene
   Pinene
   Menthol
   Menthone
   Zingiberene
   Sesquiphellandrene
   Betacaryophyllene
Sodium benzoate
Potassium sorbate
Anhydrous citric acid taken from a natural source as pH regulator
Optionally a high-methoxylated rapid citrus pectin.
A surfactant—or mixture of surfactants—to regulate Hydrophilic-lipophilic balance (HLB).
Optionally, organic acids selected from fumaric, formic, propionic, lactic, butyric or a mixture thereof.

Wherein the products are formulated in the following quantities:
45-55% starch
35-45% water
0-0.1% alpha-amylase
5-15% mixture of essential oils
0.01-0.10% benzoate
0.01-0.10% sorbate
0-0.05% citric acid
0-0.5% citrus pectin
0-10% organic acids selected from fumaric, formic, propionic, lactic, butyric, or a mixture thereof

and comprising the following stages:
Introducing 100% of the water into a reactor
Start and keep stirring, during the process, in a range between 95 and 105 rpm.
Add between 40 and 45% starch
Add between 40 and 45% of the alpha-amylase
Increase the temperature to a range between 45 and 55° C. The starch gelation process starts in these temperature ranges, changing the physicochemical characteristics of the mixture, slowing down the heat exchange.
Maintain the temperature at 50° C. for 10 to 25 minutes for the gelation process to consolidate.
Bring the temperature to a range between 65 and 75° C.
Take 3 to 10% of the mixture present in the reactor outlet duct, remove it from the equipment and add it back to the reactor to impregnate and achieve a greater dispersion of the substrate and the enzyme.
Bring the temperature to a range between 85 and 95° C. to achieve maximum efficiency in the enzymatic activity of alpha-amylase.
Take 3 to 10% of the mixture present in the reactor outlet duct, remove it from the equipment and add it back to the reactor to impregnate and achieve a greater dispersion of the substrate and the enzyme.
Maintain agitation and temperature for 10 to 25 minutes to allow enzymatic action.
Lower the temperature in the reactor to a range between 65 and 75° C. to be able to add the starch and prevent it from gelling or denaturing at this stage of the process.
Add between 40 and 45% starch
Add between 40 and 45% of the alpha-amylase
Take 3 to 10% of the mixture present in the reactor outlet duct, remove it from the equipment and add it back to the reactor to impregnate and achieve a greater dispersion of the substrate and the enzyme.
Bring the temperature to a range between 85 and 95° C. to achieve maximum efficiency in the enzymatic activity of alpha-amylase.
Take 3 to 10% of the mixture present in the reactor outlet duct, remove it from the equipment and add it back to the reactor to impregnate and achieve a greater dispersion of the substrate and the enzyme.
Maintain agitation and temperature for 10 to 25 minutes to allow enzymatic action.
Lower the temperature in the reactor to a range between 65 and 75° C. to be able to add the starch and prevent it from gelling or denaturing at this stage of the process.
Add the remaining formulated starch.
Add the remaining formulated alpha-amylase.
Take 3 to 10% of the mixture present in the reactor outlet duct, remove it from the equipment and add it back to the reactor to impregnate and achieve a greater dispersion of the substrate and the enzyme.
Bring the temperature to a range between 85 and 95° C. to achieve maximum efficiency in the enzymatic activity of alpha-amylase.
Take 3 to 10% of the mixture present in the reactor outlet duct, remove it from the equipment and add it back to the reactor to impregnate and achieve a greater dispersion of the substrate and the enzyme.
Maintain agitation and temperature for 10 to 25 minutes to allow enzymatic action.
Add the formulated acids.
Add the benzoate and sorbate.
Keep stirring for another 5 to 15 minutes.
Stop stirring.
Send the product to a new reactor.
Start stirring at between 3000 and 4000 rpm if a microemulsion is desired or between 4000 and 5000 rpm if a nanoemulsion is desired.
Add the essential oil or mixture of formulated essential oils.
Add the formulated pectin in such a way as to increase the stability of the formulation.
Continue homogenization with stirring between 3000 and 4000 rpm.
Introduce the product into a spray dryer at a temperature between 40 and 70° C.
Introduce the drying air at a temperature between 160 and 220° C.
Control the outlet temperature so that the product exits at a humidity between 5 and 10%. These ranges usually fluctuate between 100 and 120° C.
Obtain the product.

### EXEMPLARY EMBODYMENTS

### Process 1

The process was carried out with the following formulation:
Cassava Starch (natural Cassava Starch) 52.34%
Water40.71%
α-Amylase 0.06%
Sodium benzoate 0.05%
Potassium Sorbate 0.05%
Anhydrous Citric Acid 0.03%
Oil composition 6.51%
Pectin 0.24%

Wherein the oil composition includes:
Thymol 54.50%
Carvacrol 15%
p-cymene 10%
γ-Terpinene 5%
Thymyl acetate 4.80%
β-myrcene 2.80%
Trans-ß-caryophyllene 2.40%
Methyl thymyl ester 1.90%

### Process 2

The process was carried out with the following formulation:
Cassava Starch (Natural Cassava Starch) 49.03%
Water38.14%
α-Amylase 0.06%
Sodium benzoate 0.05%
Potassium Sorbate 0.04%
Anhydrous Citric Acid 0.03%
Oil composition 12.20%
Pectin 0.46%

Wherein the oil composition includes:
Thymol 54.50%
Carvacrol 15%
p-cymene 10%
γ-Terpinene 5%
Thymyl acetate 4.80%
β-myrcene 2.80%
Trans-ß-caryophyllene 2.40%
Methyl thymyl ester 1.90%

1. A composition of matter for the production of micro or nanoencapsulated essential oils comprising:
A starch having amylose concentrations between 60% and 80% and amylopectin concentrations between 20% and 40%;
35-45% water;
5-15% of at least one essential oil;
An alpha amylase allowing an α-1-4 enzymatic hydrolysis between 0 and 0.1%;
0.01-0.10% sodium benzoate;
0.01-0.10% potassium sorbate;
0 and 0.05% anhydrous citric acid obtained from a natural source;
0-0.5% high-methoxylated rapid citrus pectin;
at least a surfactant; and
0-10% organic acid, selected from the group consisting of fumaric, formic, propionic, lactic, butyric, and a mixture thereof.

2. The composition according to claim 1, wherein the starch is selected from the group consisting of:
Cassava starch containing between 70%-80% amylose and 20%-30% amylopectin;
Corn and cassava starch containing between 65%-75% amylose and 25%-35% amylopectin;
Potato starch containing between 70%-80% amylose and 20-30% amylopectin;
Wheat starch containing 70%-75% amylose and 25%-30% amylopectin;
Rice starch containing between 60%-80% amylose and 20%-40% amylopectin; and
Green banana starch between 70%-75% amylose and 25%-30% amylopectin.

3. Composition according to claim 1, wherein the essential oils includes at least one of the compounds selected from the group consisting of:
a. Eugenol;
b. Betacaryophyllene;
c. Thymol;
d. p-cymene;
e. γ-Terpinene;
f. Thymyl acetate;
g. ß-myrcene;
h. Trans-ß-Caryophyllene;
i. Methyl thymyl ester;
j. Carvacrol;
k. p-cymene;
l. Geranial;
m. Neral;
n. Geraniol;
o. 1.8 cineol;
p. Limonene;
q. Pinene;
r. Menthol;
s. Menthone;
t. Zingiberene;
u. Sesquiphellandrene; and
v. Betacaryophyllene.

4. A process for the production of micro or nanoencapsulated essential oils comprising:
a starting composition containing:
45-55% starch;
35-45% water;
0-0.1% alpha-amylase;
5-15% oil or mixture of essential oils;
0.01-0.10% benzoate;
0.01-0.10% sorbate;
0-0.05% citric acid;
0-0.5% citrus pectin;
0-10% organic acids selected from the group consisting of at least one of fumaric, formic, propionic, lactic, butyric, and a mixture thereof;
and comprising the following steps:
Introducing 100% of the water into a reactor, wherein the reactor contains an reactor outlet duct;
stirring the water in the reactor in a range between 95 and 105 rpm;
Adding between 40 and 45% starch;
Adding between 40 and 45% of the alpha-amylase;
Adjusting the temperature to a range between 45 and 55° C.
Maintaining the temperature at 50° C. for 10 to 25 minutes for the gelation process to consolidate;
Adjusting the temperature to a range between 65 and 75° C.;
Removing 3 to 10% of the mixture present in the reactor outlet duct, remove it from the equipment and adding it back to the reactor;
Bringing the temperature to a range between 85 and 95° C.;
Removing 3 to 10% of the mixture present in the reactor outlet duct, remove it from the equipment and adding it back to the reactor;
Maintaining agitation and temperature for 10 to 25 minutes;
Lowering the temperature in the reactor to a range between 65 and 75° C.;
Adding between 40 and 45% starch;
Adding between 40 and 45% of the alpha-amylase;
Removing 3 to 10% of the mixture present in the reactor outlet duct, remove it from the equipment and adding it back to the reactor;
Bringing the temperature to a range between 85 and 95° C.;
Removing 3 to 10% of the mixture present in the reactor outlet duct, remove it from the equipment and adding it back to the reactor;
Maintaining agitation and temperature for 10 to 25 minutes;
Lowering the temperature in the reactor to a range between 65 and 75° C.;
Adding the remaining formulated starch;
Adding the remaining formulated alpha-amylase;
Removing 3 to 10% of the mixture present in the reactor outlet duct, remove it from the equipment and adding it back to the reactor;
Adjusting the temperature to a range between 85 and 95° C.;
Removing 3 to 10% of the mixture present in the reactor outlet duct, remove it from the equipment and adding it back to the reactor;
Maintaining agitation and temperature for 10 to 25 minutes;
Adding the formulated acids;
Adding the benzoate and sorbate;
Stirring for another 5 to 15 minutes;
Stoping stirring;
Sending the product to a second reactor;
Stirring at between 3000 and 4000 rpm if a microemulsion is desired or between 4000 and 5000 rpm if a nano emulsion is desired;
Adding the essential oil or mixture of formulated essential oils;
Adding the formulated pectin;
Continue stirring between 3000 and 4000 rpm;
Introducing the product into a spray dryer at a temperature between 40 and 70° C.;
Introducing the drying air into the spraying unit at a temperature between 160 and 220° C.; and
Controling the outlet temperature so that the product exits at a humidity between 5 and 10%.

5. The process according to claim 4, wherein the essential oils includes at least one of the compounds selected from the group consisting of:
a. Eugenol;
b. Betacaryophyllene;
c. Thymol;
d. p-cymene;
e. γ-Terpinene;
f. Thymyl acetate;
g. ß-myrcene;
h. Trans-ß-Caryophyllene;
i. Methyl thymyl ester;
j. Carvacrol;
k. p-cymene;
l. Thymol;
m. Geranial;
n. Neral;
o. Geraniol;
p. 1.8 cineol;
q. Limonene;
r. α pinene;
s. Menthol;
t. Menthone;
u. Zingiberene;
v. Sesquiphellandrene; and
w. Betacaryophyllene.

6. The encapsulated essential oil obtained through the the process of claim 4.

7. The encapsulated essential oil obtained through the the process of claim 5.

8. The encapsulated essential oil according to claim 6, wherein it is used in animal feed.

9. The encapsulated essential oil according to claim 7, wherein it is used in animal feed.

10. The encapsulated essential oil according to claim 6, wherein it is used to feed poultry.

11. The encapsulated essential oil according to claim 7, wherein it is used to feed poultry.

12. The encapsulated essential oil according to claim 6, wherein it is used to feed pigs or cattle.

13. The encapsulated essential oil according to claim 7, wherein it is used to feed pigs or cattle.
